(19) United States
(12) Patent Application Publication
(10) Pub. No.: US 2008/0234294 A1
(43) Pub. Date: Sep. 25, 2008

(54) Cxcr4 Binding Molecules

(75) Inventors: Jiri Kovarik, Zurich (CH); Gebhard Thoma, Lorrach (DE); Beat Weidmann, Winchester, MA (US); Timothy Wright, Swampscott, MA (US); Hans-Gunter Zerwes, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(73) Assignee: Novartis AG

(21) Appl. No.: 12/064,068

(22) PCT Filed: Aug. 17, 2006
(86) PCT No.: PCT/US2006/032192

§ 371 Date: Feb. 18, 2008

Related U.S. Application Data
(60) Provisional application No. 60/709,399, filed on Aug. 18, 2005.

(51) Int. Cl. A61K 31/519; A61K 31/155; A61P 37/00

(52) U.S. Cl. 514/259.1; 514/636

(57) ABSTRACT

This invention relates to the use of a CXCR binding molecules as described in the specification, in WHIM syndrome.

The present invention relates to the use of a CXCR4 binding molecules in the treatment or prevention of WHIM and diseases associated thereof, as well as in hematopoietic stem cell or progenitor cells mobilization and tissue regeneration.

WHIM (Warts Hypogammaglobulinemia Immunideficiency Myelokathexis) is a genetic syndrome characterized by neutropenia associated with bone marrow hypercellularity (myelokathexis), hypogammaglobulinemia and extensive human papillomavirus (HPV) infection. That syndrome is an hematological disorder in which mature neutrophils fail to exit the bone marrow and B- and T-cell abundance or function is variably deficient (lymphopenia). Lymphoproliferative disease and lymphoma can be a complication of WHIM syndrome.

Blood cells play a crucial part in maintaining the health and viability of animals, including humans. White blood cells include neutrophils, macrophage, eosinophils and basophils/mast cells as well as the B- and T-cells of the immune system. White blood cells are continuously replaced via the hematopoietic system, by action of colony stimulating factors (CSF) and various cytokines on stem cells and progenitor cells in hematopoietic tissues. Recent data support a model in which myelokathexis may result from impaired neutrophil migration from bone marrow.

CXCR4 is a chemokine receptor G protein coupled receptor (GPCR) that is expressed in a variety of normal tissues, including leukocytes. SDF-1 (CXCL12) is the cognate ligand of this receptor, and is known to act as a chemoattractant that drives chemotaxis of cells expressing CXCR4. Mutations in the CXCR4 gene have been associated with WHIM syndrome. Furthermore functional alterations of CXCR4-medited responses constitute a common biological trait of this pathology.

Accordingly, there is a need for agents which are effective in hematopoietic stem cell mobilization and in the treatment of WHIM syndrome.

It has been found that the molecules antagonizing CXCR4 which are described hereinbelow, can be used to prevent, treat or limit WHIM syndrome or the diseases associated with that syndrome, e.g. to prevent, treat or limit HPV infection.

Furthermore it has been found that these CXCR4 binding molecules are particularly effective in increasing white blood cells count, progenitor cells and/or stem cells and in mobilizing hematopoietic stem cells.

The present invention provides the use of isothiourea derivatives in preventing or treating WHIM, or in mobilizing hematopoietic stem cells or in tissue regeneration, wherein the isothiourea derivatives are of formula I

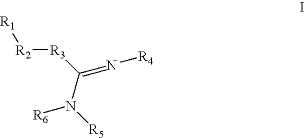

wherein
R₁ is a residue of formula (a), (b) or (c)

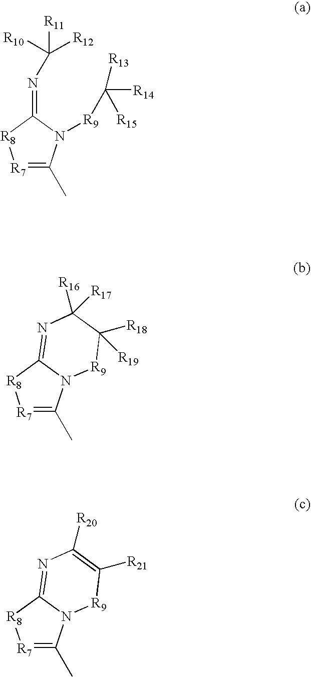

### R₂ is —(CR₂₂R₂₃)₁₋₃- or —C(O)—;

each of R₃ and R₈ independently is S; O; or NR₂₄;
each of R₄ and R₅ independently is optionally R₂₅-substituted C₃-C₁₂ cycloalkyl, C₁-C₁₂ alkyl or saturated C₈₋₁₂ polycyclic residue; or optionally R₂₆- and/or R₂₇-substituted aryl, arylC₁₋₄alkyl or heteroaryl; wherein up to 4 carbon atoms of R₄ and/or R₅ are optionally substituted by S, O or NR₂₄;
R₆ is H; C₁-C₆ alkyl; C₃-C₆ cycloalkyl; or optionally R₂₆- and/or R₂₇-substituted aryl, arylC₁₋₄alkyl or heteroaryl;

### R₇ is CR₂₈ or N;

R₉ is a direct bond; —(CR₂₂R₂₃)₁₋₂—; or NR₂₄;
each of R₁₀₋₂₃ and R₂₈ independently is H; F; Cl; Br; C₁-C₆ alkyl; C₂-C₆ alkoxyalkyl; C₁-C₆ halogenoalkyl; C₃-C₆ cycloalkyl; optionally R₂₆— and/or R₂₇-substituted aryl or heteroaryl; CONR₂₉R₃₀; COOR₂₉; CN; NO₂; or OR₃₁; or
two of R₁₀₋₁₉ which are attached to the same carbon atom, together with the carbon atom to which they are attached, form a 3-7 membered nonaromatic ring optionally containing up to two heteroatoms selected independently from N, O and S; or
R₁₇ and R₁₈, together with the C atoms to which they are attached, form a 4-7 membered nonaromatic ring optionally containing up to two heteroatoms selected independently from N, O and S; or
R₂₀ and R₂₁, together with the carbon atoms to which they are attached, form an optionally R₂₆- and/or R₂₇-substituted aryl or heteroaryl;
each of R₂₄, R₂₉ and R₃₀ independently is H; C₁-C₆ alkyl; C₂-C₆ alkoxyalkyl; C₁-C₆ halogenoalkyl; C₃-C₇ cycloalkyl; or optionally R₂₆- and/or R₂₇-substituted aryl, arylC₁₋₄alkyl or heteroaryl;
R₂₅ represents 1 to 4 substituents each independently having one of the significances given for R₁₀₋₂₃ above;
R₂₆ represents 1 to 4 substituents each independently selected from C₁-C₆ alkyl; C₁-C₆ hydroxyalkyl; C₂-C₆ alkoxyalkyl; C₁-C₆ halogenoalkyl; C₃-C₆ cycloalkyl; C₂-C₆ alkenyl; C₃-C₆ cycloalkenyl; C₂-C₆ alkynyl; aryl; heteroaryl; heteroaryl N-oxide; F; Cl; Br; I; OH; OR₄; CONH₂; CONHR₄; CONR₄R₄; OC(O)R₄; OC(O)OR₄; OC(O)NHR₄; OC(O)NR₄R₄; OSO₂R₄; COOH; COOR₄; CF₃; CHF₂; CH₂F; CN; NO₂; NH₂; NHR₄; NR₄R₄; NHC(O)R₄; NR₄C(O)R₄; NHC(O)NHR₄; NHC(O)NH₂; NR₄C(O)NHR₄; NR₄C(O)NR₄R₄; NHC(O)OR₄; NR₄C(O)OR₄; NHSO₂R₄; N(SO₂R₄)₂; NR₄SO₂R₄; SR₄; S(O)R₄; SO₂R₄; Si(CH₃)₃ and B(OC(CH₃)₂)₂;
R₂₇ represents two adjacent substituents which form an annulated 4-7 membered nonaromatic ring optionally containing up to two heteroatoms selected independently from N, O and S;
R₃₁ is C₁-C₆ alkyl; C₃-C₇ cycloalkyl; optionally R₂₆- and/or R₂₇-substituted aryl, arylC₁₋₄alkyl or heteroaryl; or CF₃.

Any alkyl, alkenyl or alkynyl may be linear or branched. Halogeno is F, Cl, Br or I.

By aryl is meant phenyl or naphthyl.

The polycyclic residue may be for example optionally R₂₅-substituted adamantyl, bicyclo[3,2.1]octyl or

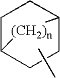

wherein n is 1 or 2.

By heteroaryl is meant an aromatic ring system comprising mono-, bi- or tricyclic systems which contains up to 4 heteroatoms independently selected from N, O and S. Examples of heteroaryl include e.g. pyridyl, indolyl, benzothiazolyl, thiazolyl, imidazolyl, benzimidazolyl. Examples of 3 to 7 membered nonaromatic rings containing 1 or 2 heteroatoms include e.g. morpholinyl, piperazinyl, piperidyl.

The compounds of formula I may exist in form of several interconverting tautomers and E/Z isomers, e.g.

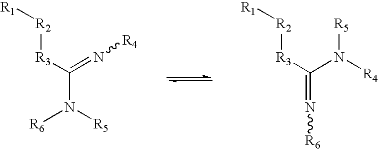

They may exist in free form or in salt form, e.g. addition salts with e.g. organic or inorganic acids, for example, hydrochloric acid, acetic acid. When the compounds of formula I have one or more asymmetric centers in the molecule, the present invention is to be understood as embracing the various optical isomers, as well as racemates, diastereoisomers and mixtures thereof.

The compounds of formula I are known and disclosed e.g. in WO2005/085219, the content thereof being incorporated therein.

Examples of suitable compounds of formula I for the invention are:
1,3-Dicyclohexyl-2-(5,6-dihydro-imidazo[2,1-b]thiazol-3-ylmethyl)-isothiourea,
1-Cyclohexyl-3-cyclopentyl-2-(5,6-dihydro-imidazo[2,1-b]thiazol-3-ylmethyl)-isothiourea,
1-Cycloheptyl-3-cyclohexyl-2-(5,6-dihydro-imidazo[2,1-b]thiazol-3-ylmethyl)-isothiourea,
1,3-Dicycloheptyl-2-(5,6-dihydro-imidazo[2,1-b]thiazol-3-ylmethyl)-isothiourea,
1-Cyclohexyl-3-cyclooctyl-2-(5,6-dihydro-imidazo[2,1-b]thiazol-3-ylmethyl)-isothiourea,
1,3-Dicyclohexyl-2-(6,6-dimethyl-5,6-dihydro-imidazo[2,1-b]thiazol-3-ylmethyl)-isothiourea,
1,3-Dicyclooctyl-2-(5,6-dihydro-imidazo[2,1-b]thiazol-3-ylmethyl)-isothiourea and 1,3-Dicycloheptyl-2-(6,6-dimethyl-5,6-dihydro-imidazo[2,1-b]thiazol-3-ylmethyl)-isothiourea. 1,3-dicyclohexyl-2-(3-methyl-2-methylimino-2,3-dihydro-thiazol-4-ylmethyl)-isothiourea.

The isothiourea derivatives of formula I can be prepared as described e.g. in WO2005/085219, the content thereof being incorporated therein.

According to the invention there is provided:
1. Use of a compound of formula I or a pharmaceutically acceptable salt thereof in the prevention or treatment of WHIM syndrome or a manifestation associated with that syndrome, in particular neutropenia, hypogammaglobulinemia or HPV infection.
2. A method for preventing or treating WHIM syndrome or a manifestation associated with that syndrome, in particular neutropenia, hypogammaglobulinemia or HPV infection, in a subject in need of such a treatment, which method comprises administering to said subject an effective amount of a compound of formula I or a pharmaceutically acceptable salt thereof.
3. Use of a compound of formula I or a pharmaceutically acceptable salt thereof in hematopoietic stem cell or progenitor cell mobilization, or for the prevention or treatment of a disease associated with such a mobilization, e.g. in tissue regeneration.
4. A method for increasing white blood cell counts, progenitor cells and/or stem cells, for mobilizing hematopoietic stem cells or progenitor cells or for regenerating tissue in a subject in need of such a method, which method comprises administering to said subject an effective amount of a compound of formula I or a pharmaceutically acceptable salt thereof.
5. A pharmaceutical composition, e.g. for use in a method as in 2 or 4 above comprising a compound of formula I or a pharmaceutically acceptable salt thereof in association with a pharmaceutically acceptable diluent or carrier therefor.
6. A compound of formula I or a pharmaceutically acceptable salt thereof for use in the preparation of a pharmaceutical composition for use in the method as in 2 or 4 above.

For the above uses the required dosage will of course vary depending on the mode of administration, the particular condition to be treated and the effect desired. In general, satisfactory results are indicated to be obtained systemically at daily dosages of from about 0.01 to 10 mg/kg per body weight. An indicated daily dosage in the larger mammal, e.g. humans, is in the range from about 0.5 mg to about 1000 mg, conveniently administered, for example, in divided doses up to four times a day or in retard form. Suitable unit dosage forms for oral administration comprise from 0.1 to 500 mg, e.g. from ca. 0.5 to 4 mg active ingredient.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula I in free form or in pharmaceutically acceptable salt form in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form e.g. as indicated above. Such salts may be prepared in conventional manner and exhibit the same order of activity as the free compounds.

For the purpose of the invention, the compounds of formula I may be administered as the sole active ingredient or in conjunction with, e.g. as an adjuvant to, other drugs e.g. for the treatment or prevention of WHIM, e.g. anti-infective or anti-inflammatory agent. For example, the compounds of formula I may be used in combination with gammaglobulin, immunoglobulin, cytokines e.g. G-CSF, GM-CSF, IL-3, stem cell factor, flt-3 ligand or with another CXCR4 antagonist, e.g. CXCR4 inhibitor or binding molecule, e.g. as described in US 2005/0043367, the content of which being enclosed herein by reference.

For the purpose of the invention, the compounds of formula I may also be administered together with other drugs effective in infectious diseases, such as antibiotics, antibacterial agents or antiviral compounds, e.g. anti HPV agent, anti HIV agent. In particular, the compounds of formula I may be administered together with Maraviroc (UK 427857) from Pfizer; Vicriviroc (SCH-417690, SCH-D), GSK (Ph 11b), GSK's 873140 (also known as AK 602 or ONO 4128) from Schering-Plough's, TAK-652 from Takeda. The compounds of formula I may also be administered together with β-lactams e.g. penicillins; cephalosporins; carbapenems; ketolides; quinolones e.g. fluoroquinolones; macrolides e.g. clarithromycin, azithromycin or vancomycin; rifamycins; monobactams; isoniazid; licosamides; mupirocin; sulfonamides; phenicols; fosfomycin; glycopeptides; tetracyclines; streptogramins; chloramphenicol; and oxazolidinone, famciclovir or penciclovir

The term “anti-viral agent” as used herein includes, but is not limited to, anti-retroviral agent; antibody against virus; e.g. anti-HIV antibody; inhibitor of reverse transcriptase; e.g. inhibitor of HIV reverse transcriptase, especially nucleoside analogues, such as Retrovir® (3′-azido-3′-deoxypyrimidine, Zidovudine) and 3′-azido-3′-deoxythymidine (AZT) from GlaxoSmithKline, HIVID® (2′,3′-dideoxycytidine, Zalcitabine) from Hoffmann-LaRoche, Videx® or VidexEC® (2′,3′dideoxyinosine, Didanosine) from Bristol-Myers-Squibb, Epivir® (Lamivudine) from GlaxoSmithKline, Zerit® (stavudine) from Bristol Myers-Squibb, Viread® (tenofovir DF) from Gilead, Ziagen® (abacavir) from GlaxoSmithKline, Emtriva® (Emtricitabine, FTC) from Gilead Sciences; or non-nucleoside analogues, such as e.g. Rescriptor® (delavirdine) from Pfizer, Sustiva® (Efavirenz) from Bristol Meyer Squibb, Viramune® (nevirapine) from Boehringer-Ingelheim; 11-cyclopropyl-5,11-dihydro-4-methyl-(6H)-dipyrido[3,2-b;2′,3′-e]-[1,4]diazepin-6-one, trisodium phosphonoformate, ammonium-21-tungstenato-9-antimonate, 1-β-D-ribofuranoxy-1,2,4-triazole-3-carboxamide; inhibitor of viral or retroviral protease, e.g. inhibitor of viral aspartate protease, e.g. inhibitor of HIV protease, such as Aganerase® (amprenavir) fromGlaxoSmithKline, Reyataz® (atazanavir) from Bristol-Myers Squibb, Lexiva® (fosamprenavir) from GSK, Crixivan ((Indinavir) from Merck & Co.; Viracept® (nelfinavir) from Agouron, Norvir® (Ritonavir) from Abbott; Fortovase® and Invirase® (saquinavir) from Hoffmann-LaRoche; and other compounds such as lasinavir (5(S)-(tert-butoxycarbonylamino)-4(S)-hydroxy-6-phenyl-2(R)(2,3,4-trimethoxyphenylmethyl)-hexanoyl-(L)-valyl-N-(2-metoxy-ethyl)-amide), Adriamycin, KVX-478 from GlaxoWellcome; VX-478 from Vertex; 141W94 from Kissei Pharmaceuticals; AG-1343 from Agouron; KNI-272 from Nippon Mining; U-96988 from Upjohn; BILA-2011 BS (Palinavir) from Boehringer-Ingelheim; compounds preventing virus penetration, such as e.g. polymannoacetate; fusion inhibitors, such as e.g. Fuzeon® (enfuvirtide, T-20) from Hofffmann-LaRoche; or any combination thereof, such as Epzicom® (Abacavir and Lamivudine) from GlaxoKlineSmith, Trizivir® (Abacavir, Lamivudine and Zidovudine) from GlaxoKlineSmith, Truvada® (Emtricitabine and Tenofir DF) from Gilead Sciences, Combivir® (Lamivudine and Zidovudine) from GlaxoKlineSmith, Kaletra® (lopinavir and ritohavir) from Abbott. The term “anti-viral agent” further includes agent which treats the opportunistic infectious which are caused by the immunosuppression resulting from viral infection, e.g. HIV infection.

The term “HIV” as used herein includes, but is not limited to, HIV-1 and HIV-2.

For the purpose of the invention, the compounds of formula I may also be administered together with a chemotherapeutic agent, e.g. an anti-retroviral agent other drugs effective in immunosuppressive or immunomodulating regimens. e.g. for the treatment or prevention of allo- or xenograft acute or chronic rejection. For example, the compounds of formula I may be used in combination with a JAK3 inhibitor, a calcineurin inhibitor, e.g. cyclosporin A or FK 506; an mTOR inhibitor, e.g. rapamycin, 40-O-(2-hydroxyethyl)-rapamycin, CC1779, ABT578, AP23573, AP23464, AP23675, AP23841 or TAFA-93; an ascomycin having immunosuppressive properties, e.g. ABT-281, ASM981, etc.; corticosteroids; cyclophosphamide; azathioprine; methotrexate; leflunomide; mizoribine; mycophenolic acid; mycophenolate mofetil; 15-deoxyspergualine or an immunosuppressive homologue, analogue or derivative thereof; a sphingosine-1-phosphate receptor agonist, e.g. FTY720; monoclonal antibodies to leukocyte receptors, e.g., MHC, CD2, CD3, CD4, CD7, CD8, CD11a/CD18, CD25, CD27, CD28, CD40. CD45, CD58, CD80, CD86, CD137, ICOS, CD150 (SLAM), OX40, 4-1 BB or to their ligands, e.g. CD154, or antagonists thereof; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or antichemokine antibodies or antichemokine receptor antibodies or low molecular weight chemokine receptor antagonists, e.g. anti MCP-1 antibodies.

A compound of formula I may also be used to advantage in combination with other antiproliferative agents. Such antiproliferative agents include, but are not limited to aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, mTOR inhibitors, antineoplastic antimetabolites, platin compounds, compounds decreasing the protein kinase activity and further anti-angiogenic compounds, gonadorelin agonists, anti-androgens, bengamides, bisphosphonates, antiproliferative antibodies and temozolomide (TEMODAL®).

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETEN™.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is mar-keted, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “histone deacetylase inhibitors” relates to compounds which inhibit the histone deacetylase and which possess antiproliferative activity.

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enyzme (COX-2) and which possess antiproliferative activity such as celecoxib (Celebrex®), rofecoxib (Vioxx®) and lumiracoxib (COX189).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “antineoplastic antimetabolites” includes, but is not limited to 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOLM) and OGT719.

The term “platin compounds” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity and further anti-angiogenic compounds” as used herein includes, but is not limited to compounds which decrease the activity of e.g. the Vascular Endothelial Growth Factor (VEGF), the Epidermal Growth Factor (EGF), c-Src, protein kinase C, Platelet-derived Growth Factor (PDGF), Bcr-Abl tyrosine kinase, c-kit, Flt-3 and Insulin-like Growth Factor I Receptor (IGF-IR) and Cyclin-dependent kinases (CDKs), and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula I), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21,1999; in WO 00/37502 and WO 94/10202; Angiostatin™, described by M. S. O'Reilly et al, Cell 79, 1994, 315-328; and Endostatin™, described by M. S. O'Reilly et al, Cell 88, 1997, 277-285;

compounds which decrease the activity of EGF are especially compounds which inhibit the EGF receptor, especially the tyrosine kinase activity of the EGF receptor, and compounds binding to EGF, and are in particular those compounds generically and specifically disclosed in WO 97/02266 (describing compounds of formula IV), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980;
compounds which decrease the activity of c-Src include, but are not limited to, compounds inhibiting the c-Src protein tyrosine kinase activity as defined below and to SH2 interaction inhibitors such as those disclosed in WO97/07131 and WO97/08193;
compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706;
compounds which decreases the activity of the protein kinase C are especially those staurosporine derivatives disclosed in EP 0 296 110 (pharmaceutical preparation described in WO 00/48571) which compounds are protein kinase C inhibitors;
further specific compounds that decrease protein kinase activity and which may also be used in combination with the compounds of the present invention are Imatinib (Gleevec®/Glivec®), midostaurin, Iressa™ (ZD1839), PKI166, Vatalanib, ZD6474, GW2016, CHIR-200131, CEP-7055/CEP-5214, CP-547632 and KRN-633;
anti-angiogenic compounds having another mechanism of action than decreasing the protein kinase activity include, but are not limited to e.g. thalidomide (THALOMID), celecoxib (Celebrex), SU5416 and ZD6126.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™.

Abarelix can be formulated, e.g. as disclosed in U.S. Pat. No. 5,843,901.

The term “anti-androgens” as used herein includes, but is not limited to bicalutamide (CASODEXT™), which can be formulated, e.g. as disclosed in U.S. Pat. No. 4,636,505.

The term “bengamides” relates to bengamides and derivatives thereof having aniproliferative properties.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

The term “antiproliferative antibodies” as used herein includes, but is not limited to trastuzumab (Herceptin™), Trastuzumab-DM1, erlotinib (Tarceva™), bevacizumab (Avastin™), rituximab (Rituxan®), PRO64553 (anti-CD40) and 2C4 Antibody.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of formula I, can be prepared and administered as described in the art such as in the documents cited above.

Where the compounds of formula I are administered in conjunction with gammaglobulin, immunoglobulin, cytokine, anti-inflammatory agent, anti-infective agent, e.g. anti-viral agent or antibiotic, or another CXCR4 antagonist, dosages of the co-administered compound will of course vary depending on the type of co-drug employed, on the specific drug employed, on the severity of the condition being treated and so forth.

In accordance with the foregoing the present invention provides in a yet further aspect:
7. A method as defined above comprising co-administration, e.g. concomitantly or in sequence, of a therapeutically effective non-toxic amount of a compound of formula I and at least a second drug substance, e.g. a gammaglobulin, an immunoglobulin, a cytokine, e.g. G-CSF, GM-CSF, IL-3, an anti-inflammatory agent, an anti-infective agent, e.g. anti-viral agent or antibiotic, a chemotherapeutic agent, e.g. an anti-retroviral agent, an antiproliferative agent, a drug effective in immunosuppressive or immunomodulating regimens, or another CXCR4 antagonist, e.g. as indicated above.
8. A pharmaceutical combination, e.g. a kit, comprising a) a first agent which is a CXCR4 antagonist, e.g. CXCR4 inhibitor or binding molecule, e.g. a compound of formula I as disclosed herein, in free form or in pharmaceutically acceptable salt form, and b) at least one co-agent, e.g. a gammaglobulin, an immunoglobulin, a cytokine, e.g. G-CSF, GM-CSF, IL-3, an anti-inflammatory agent, an anti-infective agent, e.g. anti-viral agent or antibiotic, a chemotherapeutic agent, e.g. an anti-retroviral agent, an antiproliferative agent or a drug effective in immunosuppressive or immunomodulating regimens. The kit may comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a co-agent, e.g. viral agent, are both administered to a patient simultaneously in the form of a single entity or dosage. The term “non-fixed combination” means that the active ingredients, e.g. a compound of formula I and a co-agent, e.g. viral agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

Utility of the CXCR4 binding molecule, e.g. compound of formula I, in the context of the invention as hereinabove specified, may be demonstrated in animal test methods as well as in clinic, for example in accordance with the methods hereinafter described.

A) In vitro

The effects of the CXCR4 binding molecule, e.g. compound of formula I, may be tested in models which reproduce the pathological exacerbated CXCL12/CXCR4-dependent migration (chemotaxis) observed in primary cells from WHIM patients. The effects of the CXCR4 binding molecule, e.g. compound of formula I, on the chemokine receptor internalization induced by CXCL12 can also be analyzed.
A.1) Chemotaxis is performed as described previously (Balabanian et al, Blood, 2005; Blood, 15 Mar. 2005, volume 105, No 6, p 2449-2457; the content being enclosed herewith) using a Transwell assay upon induction with CXCL12. Briefly, 3×10⁵ cells in 150 μL RPMI medium supplemented with 20 mM HEPES and 1% human AB serum is added to the upper chamber of a 6.5-mm diameter, 5 □m pore polycarbonate Transwell culture insert. 600 μL of the same medium with or without chemokine is placed in the lower chamber. Chemotaxis proceeds for 2 h at 37° C. in humidified air with 5% CO₂. CXCL12 is used at 30 nM. Transmigrated cells recovered in the lower chamber are counted by flow cytometry with gating on forward and side scatter and the fraction of migrating cells is calculated as follows: {[(number of cells migrating to the lower chamber in response to chemokine)−(number of cells migrating spontaneously)]/number of cells added to the upper chamber at the start of the assay}×100. Flow cytometry analysis is carried out on a FACSCalibur® using the anti-human CXCR4 (clone 12G5) mAbs (from Becton Dickinson). Background fluorescence is evaluated using the corresponding PE-conjugated, immunoglobulin-isotype control mAb.
A.2) The inhibitory potency of the CXCR4 binding molecule on the internalization process of CXCRR4 in response to CXCL12 is investigated in circulating T lymphocytes from healthy subjects and in WHIM patients, as described previously (Balabanian et al, Blood, 2005; Blood, 15 Mar. 2005, volume 105, No 6, p 2449-2457; the content being incorporated therein). Cells are incubated with CXCR4 binding molecule and CXCL12 at 37° C. After a wash in acidic glycin buffer (pH=2.7), levels of receptor cell surface are determined using CXCR412G5 mAb (PE-conjugated). Background fluorescence is evaluated using the corresponding PE-conjugated, immunoglobulin-isotype control mAb. Receptor expression in stimulated cells is calculated as follows: (receptor geometric mean fluorescence intensity of treated cells/receptor geometric mean fluorescence intensity of unstimulated cells)×100. Receptor internalization is deduced after substracting receptor expression values from that of control cells (cell incubated in medium alone), which is arbitrarily set at 100%.

### B) Clinical Trial

A person suffering from WHIM may exhibit one or more of the following signs or symptoms: (a) Warts (b) Hypogammaglobulinemia (c) Immunideficiency, in particular human papillomavirus (HPV) infection, (d) Myelokathexis (d) neutropenia (e) lymphopenia. Such criteria may not only be used to diagnose WHIM, but can be used to evaluate a patient's response to drug treatment.

The efficacy of the treatment will be assessed based on normalization of parameters such as peripheral blood cell counts (e.g. granulocytes, lymphocytes e.g. B cells, platelets), reduction of bone marrow hypercellularity, normalization of blood immunoglobulin levels, decrease of the rate of infection.

Blood cell counts including differentials may be determined in animals treated with compounds of formula I by using standard haematological methodology e.g. whole blood cell counters.

1. A method of preventing or treating WHIM syndrome or a manifestation associated thereof comprising administering to a subject an effective amount of a molecule of formula I 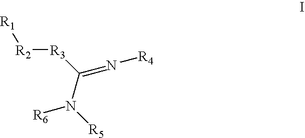
wherein
R₁ is a residue of formula (a), (b) or (c) 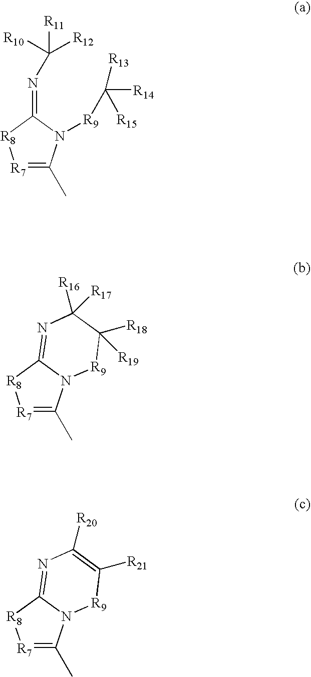
R₂ is —(CR₂₂R₂₃)₁₋₃— or —C(O)—;
each of R₃ and R₈ independently is S; O; or NR₂₄;
each of R₄ and R₅ independently is optionally R₂₅-substituted C₃-C₁₂ cycloalkyl, C₁-C₁₂ alkyl or saturated C₈₋₁₂ polycyclic residue; or optionally R₂₆- and/or R₂₇-substituted aryl, arylC₁₋₄alkyl or heteroaryl; wherein up to 4 carbon atoms of R₄ and/or R₅ are optionally substituted by S, O or NR₂₄;
R₆ is H; C₁-C₆ alkyl; C₃-C₆ cycloalkyl; or optionally R₂₆- and/or R₂₇-substituted aryl, arylC₁₋₄alkyl or heteroaryl;
R₇ is CR₂₈ or N;
R₉ is a direct bond; —(CR₂₂R₂₃)₁₋₂—; or NR₂₄;
each of R₁₀₋₂₃ and R₂₈ independently is H; F; Cl; Br; C₁-C₆ alkyl; C₂-C₆ alkoxyalkyl;
C₁-C₆ halogenoalkyl; C₃-C₆ cycloalkyl; optionally R₂₆- and/or R₂₇-substituted aryl or heteroaryl; CONR₂₉R₃₀; COOR₂₉; CN; NO₂; or OR₃₁; or
two of R₁₀₋₁₉ which are attached to the same carbon atom, together with the carbon atom to which they are attached, form a 3-7 membered nonaromatic ring optionally containing up to two heteroatoms selected independently from N, O and S; or
R₁₇ and R₁₈, together with the C atoms to which they are attached, form a 4-7 membered nonaromatic ring optionally containing up to two heteroatoms selected independently from N, O and S; or
R₂₀ and R₂₁, together with the carbon atoms to which they are attached, form an optionally R₂₆- and/or R₂₇-substituted aryl or heteroaryl;
each of R₂₄, R₂₉ and R₃₀ independently is H; C₁-C₆ alkyl; C₂-C₆ alkoxyalkyl; C₁-C₆ halogenoalkyl; C₃-C₇ cycloalkyl; or optionally R₂₆- and/or R₂₇-substituted aryl, arylC₁₋₄alkyl or heteroaryl;
R₂₅ represents 1 to 4 substituents each independently having one of the significances given for R₁₀₋₂₃ above;
R₂₆ represents 1 to 4 substituents each independently selected from C₁-C₆ alkyl; C₁-C₆ hydroxyalkyl; C₂-C₆ alkoxyalkyl; C₁-C₆ halogenoalkyl; C₃-C₆ cycloalkyl; C₂-C₆ alkenyl; C₃-C₆ cycloalkenyl; C₂-C₆ alkynyl; aryl; heteroaryl; heteroaryl N-oxide; F; Cl; Br; I; OH; OR₄; CONH₂; CONHR₄; CONR₄R₄; OC(O)R₄; OC(O)OR₄; OC(O)NHR₄; OC(O)NR₄R₄; OSO₂R₄; COOH; COOR₄; CF₃; CHF₂; CH₂F; CN; NO₂; NH₂; NHR₄; NR₄R₄; NHC(O)R₄; NR₄C(O)R₄; NHC(O)NHR₄; NHC(O)NH₂; NR₄C(O)NHR₄; NR₄C(O)NR₄R₄; NHC(O)OR₄; NR₄C(O)OR₄; NHSO₂R₄; N(SO₂R₄)₂; NR₄SO₂R₄; SR₄; S(O)R₄; SO₂R₄; Si(CH₃)₃ and B(OC(CH₃)₂)₂;
R₂₇ represents two adjacent substituents which form an annulated 4-7 membered nonaromatic ring optionally containing up to two heteroatoms selected independently from N, O and S;
R₃₁ is C₁-C₆ alkyl; C₃-C₇ cycloalkyl; optionally R₂₆- and/or R₂₇-substituted aryl, arylC₁₋₄alkyl or heteroaryl; or CF₃;
or a pharmaceutically acceptable salt thereof.

2. The Method of claim 1 wherein said WHIM syndrome is neutropenia, hypogammaglobulinemia or HPV infection.

3. The Method of claim 1 wherein said WHIM syndrome is a disease associated with hematopoietic stem cell or progenitor cell mobilization, or in tissue regeneration.

4. The method of claim 1 wherein the compound of formula I is co-administered with a agent selected from gammaglobulins, immunoglobulins, cytokines, anti-inflammatory agents, anti-infective agents, chemotherapeutic agents, antiproliferative agents, immunosuppressive agents and immunomodulating agents, in particular antiviral agents, antibiotics, G-CSF, GM-CSF, IL-3, stem cell factor and flt-3 ligand.

5. The method of claim 1 wherein the compound of formula I is selected from 1,3-Dicyclohexyl-2-(5,6-dihydro-imidazo[2,1-b]thiazol-3-ylmethyl)-isothiourea; 1-Cyclohexyl-3-cyclopentyl-2-(5,6-dihydro-imidazo[2,1-b]thiazol-3-ylmethyl)-isothiourea; 1-Cycloheptyl-3-cyclohexyl-2-(5,6-dihydro-imidazo[2,1-b]thiazol-3-ylmethyl)-isothiourea; 1,3-Dicycloheptyl-2-(5,6-dihydro-imidazo[2,1-b]thiazol-3-ylmethyl)-isothiourea; 1-Cyclohexyl-3-cyclooctyl-2-(5,6-dihydro-imidazo[2,1-b]thiazol-3-ylmethyl)-isothiourea; 1,3-Dicyclohexyl-2-(6,6-dimethyl-5,6-dihydro-imidazo[2,1-b]thiazol-3-ylmethyl)-isothiourea; 1,3-Dicyclooctyl-2-(5,6-dihydro-imidazo[2,1-b]thiazol-3-ylmethyl)-isothiourea; 1,3-dicyclohexyl-2-(3-methyl-2-methylimino-2,3-dihydro-thiazol-4-ylmethyl)-isothiourea; and 1,3-Dicycloheptyl-2-(6,6-dimethyl-5,6-dihydro-imidazo[2,1-b]thiazol-3-ylmethyl)-isothiourea.

6. A pharmaceutical combination comprising a) a first agent which is a CXCR4 antagonist of formula I as defined in claim 1 or a pharmaceutically acceptable salt thereof and b) at least one active ingredient selected from the group consisting of gammaglobulins, immunoglobulins, cytokines, anti-inflammatory agents, anti-infective agents, chemotherapeutic agents, antiproliferative agents, immunosuppressive agents, antiviral agents, antibiotics and immunomodulating agents.

7. The combination according to claim 6 wherein the second active ingredient is selected from the group consisting of G-CSF, GM-CSF, IL-3, stem cell factor and flt-3 ligand.

8. A method for treating or preventing WHIM syndrome or a manifestation associated with that syndrome in a subject in need thereof, comprising administering to said subject a therapeutically effective amount of a compound of formula I or a pharmaceutically acceptable salt thereof.

9. A pharmaceutical composition comprising a compound of formula I as defined in claim 1 or a pharmaceutically acceptable salt thereof, together with one or more pharmaceutically acceptable diluents or carriers therefor.

10. A compound comprising formula I as defined in claim 1 or a pharmaceutically acceptable salt thereof.
